(19) United States
(12) Patent Application Publication
(10) Pub. No.: US 2002/0137662 A1
(43) Pub. Date: Sep. 26, 2002

(54) Compositions and methods for negative regulation of TGF-beta pathways

(76) Inventor: Allen S. Laughon, Madison, WI (US)

Correspondence Address: LICATLA & TYRRELL P.C., 66 E. MAIN STREET, MARLTON, NJ, 08053, US

(21) Appl. No.: 09/810,385

(22) Filed: Mar. 16, 2001

(51) Int. Cl. A61K 31/00; G01N 33/53; G01N 33/567

(52) U.S. Cl. 514/1; 435/7.2

(57) ABSTRACT

Methods for screening for compounds that are negative regulators of TGF-β-regulated gene expression in mammalian cells are provided, including compositions identified therefrom.

This invention was made with United States government support awarded by the following agency: NSF-9723455. The United States has certain rights in this invention.

### BACKGROUND OF THE INVENTION

The TGF-β superfamily is one of the largest groups of polypeptide growth and differentiation factors. A variety of structural and functional criteria have been used to group the superfamily into three classes: (1) TGF-β's; (2) activins; and (3) bone morphogenetic proteins (BMPs). Members of these groups mediate a wide range of biological processes in vertebrates and invertebrates, including regulation of cell proliferation, differentiation, recognition, and death, and thus play a major role in developmental processes, tissue recycling, and repair (Wrana, J. and L. Attisano. 1996. TIG 12:493-496).

TGF-β pathways constitute one of the major signal transduction mechanisms in animals, collectively affecting a myriad of processes in development, in tissue homeostasis, and in responses to injury and infection. It is clear that TGF-β pathways play an important role in cancer and a variety of heritable disorders. The function or dysfunction of TGF-β pathways has been implicated in several major diseases, including lung cancer, prostate cancer, breast cancer, colon and gastric cancers, vascular disease, and a variety of fibrotic diseases.

Genetic and biochemical studies indicate that TGF-β and its related factors, including activin, bone morphogenetic proteins (BMPs), and their Drosophila counterpart, decapentaplegic, each signal to their target cells by a unique signaling cascade activated by ligand-induced serine/threonine kinase receptor complex formation (Wrana, T. 1998. Miner. Electrolyte Metab. 24:1201-30). It is now well established that TGF-β signaling pathways switch target genes on through the activities of Smad proteins. These cytosolic proteins are recruited and phosphorylated by the TGF-β, activin, or BMP receptor complexes. Smad proteins exist as monomers in unstimulated cells but homo- or hetero-dimerize and translocate to the nucleus of the cells where they then activate target gene expression through contact with cofactors and DNA.

Thus, although much is now known about how TGF-β pathways switch genes on, little is known about how genes can be switched off. There are examples of such negative regulation in vertebrates and in model organisms such as C. elegans and Drosophila. In mammals, growth inhibition by TGF-β is correlated with negative regulation of c-myc and cyclin A (Feng, X. H. et al. 1995. J. Biol. Chem. 270:24237-24245). TGF-β also negatively regulates proteases that degrade components of the extracellular matrix such as collagen (Kerr, L. D. et al. 1990. Cell 61:267-278). Evidence that Smad proteins can directly repress or negatively regulate transcription comes from genetic analysis of the C. elegans TGF-β pathway that regulates choice between reproductive growth and diapause (Patterson, G. I. et al. 1997. Genes Develop. 11:2679-2690). Activation of this pathway over-rides negative regulation by the Smad4-related Daf-3 protein. Negative regulation by Smad proteins was also shown in Drosophila, where the Drosophila BMP4 homolog, decapentaplegic (dpp), was shown to activate its targets by repressing expression of a novel repressor known as Brinker (Campbell, G. And A. Tomlinson. 1999. Cell 96:553-562; Jazwinska, A. Et al. 1999. Cell 96:563-573; Minami, M. Et al. 1999. Nature 398:242-246; Sivasankaran, R. Et al. 2000. EMBO J. 19:6162-6172). Ectopically expressed Brinker was able to repress BMP targets in frog embryos as well, indicating that this double negative mechanism is likely to operate in vertebrates as well as in Drosophila. A second negatively regulated target is the segment polarity gene, wingless (wg), which is repressed in response to Dpp in the embryonic ectoderm (Grieder, N. et al. 1995. Cell 81:791-800) and in imaginal discs (Penton, A. and F. M. Hoffmann. 1996. Nature 382:162-164; Brook, W. J. and S. M. Cohen. 1996. Science 273:1373-1377; Jiang, J. and G. Struhl. 1996. Cell 86:401-409; Theisen, H. et al. 1996. Development 122:3939-3948; Tomoyasu, Y. et al. 1998. Development 125:4215-4224; Chanut, F. and U. Heberlein. 1997. Development 124:559-567).

Although repression by TGF-β pathways could be indirect, mounting evidence shows that Smad proteins interact directly with a variety of co-repressors. Smad proteins interact with the repressors Evi-1 (Kurokawa, M. Et al. 1998. Nature 394:92-96), Gli3 (Liu, F. Et al. 1998. Nature Genet. 20:325-326), TGIF (Wotton, D. Et al. 1999. Cell 97:29-39), SIP1 (Verschueren, K. Et al. 1999. J. Biol. Chem. 274:20489-20498), and the oncoproteins SKI (Luo, K. S. et al. 1999. Genes Develop. 13:2196-2206), SnoN (Stroschein, S. L. et al. 1999. Science 286:771-779), and adenovirus 1A (Nishihara, A. et al. 1999. J. Biol. Chem. 274:28716-28723). It is known that some viruses inhibit cellular responses to TGF-β and the finding that E1A interacts directly with Smad proteins supports this finding. Binding of Smad3 to E1A or TGIF inhibits Smad binding of the coactivator p300. Contact with TGIF or SKI recruits histone deacetylase and inhibits transcriptional activation by Smad2 and Smad3. Contact with Evi-l inhibits DNA binding of Smad3. However, because Smad proteins are not known to have any intrinsic ability to function as repressors, and in fact have just the opposite effect, the function generally ascribed to DNA-binding Smad co-repressors is one of dampening of transcriptional activation by Smads, leaving the mechanism of TGFβ-induced repression unexplained. Until the present invention it was not appreciated that Smad proteins are able to interact directly with co-repressor genes through a specific Smad domain. Thus, the present invention describes the interaction between Smad proteins and the general co-repressor dCtBP and shows how this interaction provides a mechanism for the ability of activated Smads to directly repress transcription in response to signaling.

### SUMMARY OF THE INVENTION

An object of the present invention is a method for identifying compounds that directly interact with Smad proteins, or with Smad co-repressors, to prevent protein-protein or protein-DNA interactions required for transcriptional repression in response to TGFβ, activin, or bone morphogenetic protein signal which comprises determining changes in the level of transcription in cells, before and after addition of a test compound, wherein a decrease in the level of transcriptional repression is indicative of the ability of the test compound to interact with Smad proteins or Smad co-repressors to interfere with transcriptional repression that otherwise would occur in response to signaling by a TGFβ, activin or bone morphogenetic protein. In another embodiment, the Smad protein and CtBP interact with a co-repressor protein to produce repression of transcription wherein the co-repressor protein is selected from the group consisting of Evi-1, TGIF, SIP1 and Schnurri.

Another object of the present invention is a composition which interacts with a Smad protein to prevent activation of DNA binding and therefore prevent TGF-β-induced repression of gene expression wherein said composition is identified by the method of the present invention that determines the effect of compounds on binding of Smad to a gene that is repressed in response to binding by an activated Smad in combination with CtBP and/or a DNA-binding Smad co-repressor.

Yet another object of the present invention is a method for identifying a candidate gene that is directly and negatively regulated by TGF-β signaling pathways through a CtBP protein which comprises determining the level of gene expression of a TGF-β-regulated gene in the presence and in the absence of a CtBP protein, such that the expression of the gene is shown to be dependent on the presence of CtBP, and wherein such dependence is indicative of the ability of the candidate gene to be directly and negatively regulated by a Smad protein working in conjunction with CtBP. In one embodiment, this method includes determining the level of binding of a Smad protein to a candidate gene in the presence and absence of dCtBP.

### DESCRIPTION OF THE DRAWINGS

FIG. 1 depicts the results of a GST pull-down assay showing the binding of dCtBP to the Mad MH1 domain. In the figure, “input” means that one half of total labeled protein was used in the binding reaction; “GST” stands for that protein bound by non-fused GST control; and “CtBP” stands for protein bound by GST-dCtBP. Knirps and Knirps(m) served as a positive control and negative control, respectively. MadNLC is a full length protein, while MadLC lacks the N-terminal DNA-binding domain and MadNL lacks the C-terminal domain responsible for activation.

FIG. 2 depicts the result of a band-shift assay examining the effect of dCtBP on Smad4 binding. The values presented at the top of the figure represent nanograms of purified protein.

FIG. 3 depicts the results of experiments examining the ability of dCtBP to inhibit activation of Smad box-lacZ reporter by Mad and Medea in Drosophila cells. The data shown are the average of three S2 transfections. Each LRR repeat contained three Smad boxes arranged as AGAC GTCT GTCT.

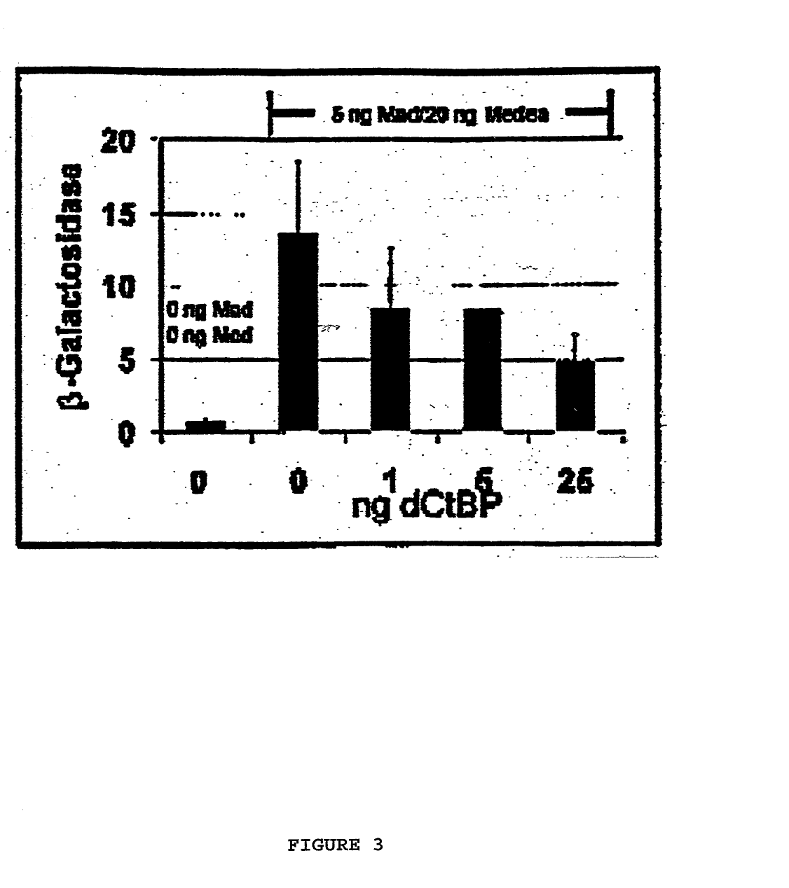

FIG. 4 depicts the results of a band-shift assay examining the interaction of Schnurri with the Mad MH2 domain. The abbreviations in the figure are the same as listed above for FIG. 1.

FIG. 5 depicts the results of experiments examining the ability of Schnurri to repress transcription of lacZ. The values listed below the bar graph represent ng of transfected effector plasmids.

FIG. 6 depicts the results of experiments examining the ability of CtBP to enhance Schnurri repression in transfected S2 cells. The values listed below the bar graph represent ng of transfected effector plasmids.

FIG. 7 depicts transcription factors with CtBP binding motifs. In the figure, vertical lines represent the CtBP motif, ovals represent zinc fingers, dashed boxes represent the homeodomain, shaded boxes represent the repression domain.

FIG. 8 depicts the alignment of CtBP motifs in various proteins. Proteins listed in bold type have been shown to bind CtBP or dCtBP.

### DETAILED DESCRIPTION OF THE INVENTION

TGF-β signaling pathways regulate transcription in cells by activating Smad proteins. Activated Smad complexes translocate to the nucleus where they activate target genes in coordination with interacting co-factors. The present invention relates to methods for screening and testing of compounds that interfere with TGFβ-dependent transcriptional repression in mammalian cells, and in cells of model organisms such as Drosophila. The screening and testing methods are based on the finding that the Drosophila Smad proteins, Mad and Medea, are able to interact directly with the co-repressor protein CtBP through the Smad MH1 domain. This was unexpected since the MH1 domain of these Smad proteins is known to lack a CtBP interaction motif or binding site. A Drosophila DNA-binding Smad co-repressor, Schnurri, has also been shown to interact both with Mad and with CtBP. In addition, an enhancer element that mediates Dpp-induced repression of the Drosophila wingless (wg) gene has been shown to be repressed by the combined expression of Schnurri and activated Mad in cultured cells. Furthermore, this repression of wg was shown to be enhanced by elevated expression of dCtBP. The data indicate that Smad proteins interact with gene-specific, DNA-bound co-repressors to cooperatively recruit CtBP which results in active repression that over-rides the positive effects of any activating transcription factors. CtBP is known to have short-range repression activity, and to interact with a histone deacetylase complex and with a component of the repressive Polycomb complex. This new mechanism is unlike previously proposed mechanisms for Smad protein interactions with co-repressor proteins, mechanisms which suggested that Smad co-repressors function to dampen transcriptional activation by Smads through (1) recruitment of histone deacetylase complex or (2) disruption of Smad interaction with the co-activator CBP. Instead, this new active repression mechanism enables TGF-β pathways to directly repress genes that are activated by other unrelated pathways or transcription factors.

In the context of the present invention, “Smad” is defined as any of the family of Smad proteins that would include and not be limited to Smad1, Smad2, Smad3, Smad4, Smad5, Smad6, and Smad7, as well as the Drosophila Smad proteins Mad and Medea. In addition, the term “Smad” would is include proteins with Smad-like functions identified in any species, including humans.

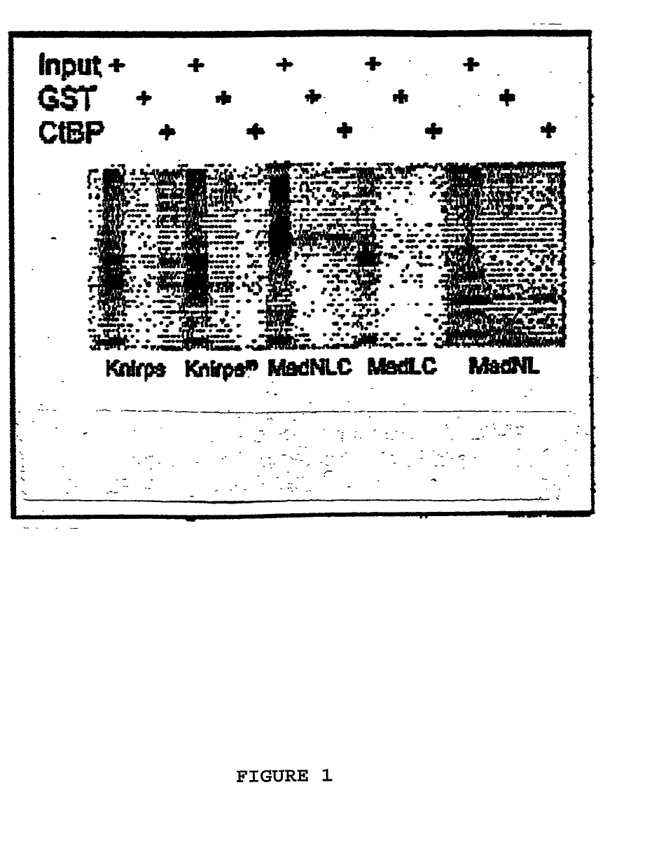

Realizing that TGF-β pathways exert direct negative regulation in addition to positive regulation is basic to understanding disease mechanisms in which TGF-β pathways are active. It has now been found that Smad proteins cooperate with a DNA-bound co-repressor, Schnurri, to recruit a second co-repressor called CtBP, dCtBP in Drosophila. Previous work has shown that dCtBP and its mammalian homologs have widespread functions as co-repressors and the ability to bind histone deacetylase. However, using radiolabeled proteins synthesized in a coupled in vitro system and a GST pull-down assay, dCtBP was shown to bind to the Mad MH1 domain (see FIG. 1). In this assay, after washing beads, bound proteins were eluted with SDS-gel loading buffer and fractionated by electrophoresis. In similar experiments, Medea was also shown to interact specifically with dCtBP.

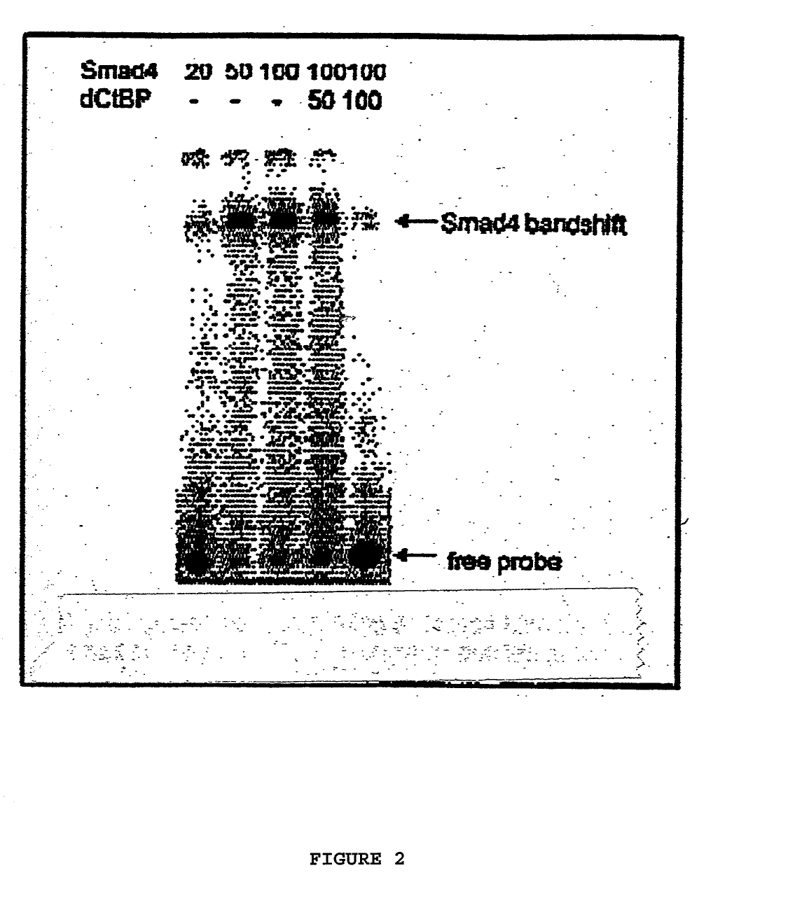

Using a band-shift assay, dCtBP was shown to be capable of disrupting the binding of Smad4 to DNA. In this assay, 100 ng of purified human Smad4 was shown to bind a radiolabeled DNA probe containing multiple Smad boxes. With addition of 100 ng of purified GST-dCtBP, that binding was almost completely abolished (see FIG. 2). Non-fused GST had no effect on binding by Smad4, in addition, GST-dCtBP had no detectable DNA binding activity of its own.

The effect of dCtBP on activation of a Smad box-lacZ reporter by Mad and Medea was then examined. Drosophila S2 cells were transfected with a reporter plasmid containing a Smad box array positioned next to a basal promoter driving lacZ. LacZ activity was determined by determining the level of β-galactosidase. LacZ activity was activated approximately 15-fold by co-transfection of plasmids expressing Mad (5 ng Mad), Medea (20 ng Medea) and an activated form of the Dpp receptor tkv. This activated expression was repressed 3-fold by co-expression of dCtBP (25 ng dCtBP). These data show that co-expression of CtBP in Drosophila S2 cells inhibited the ability of Smad proteins to activate transcription.

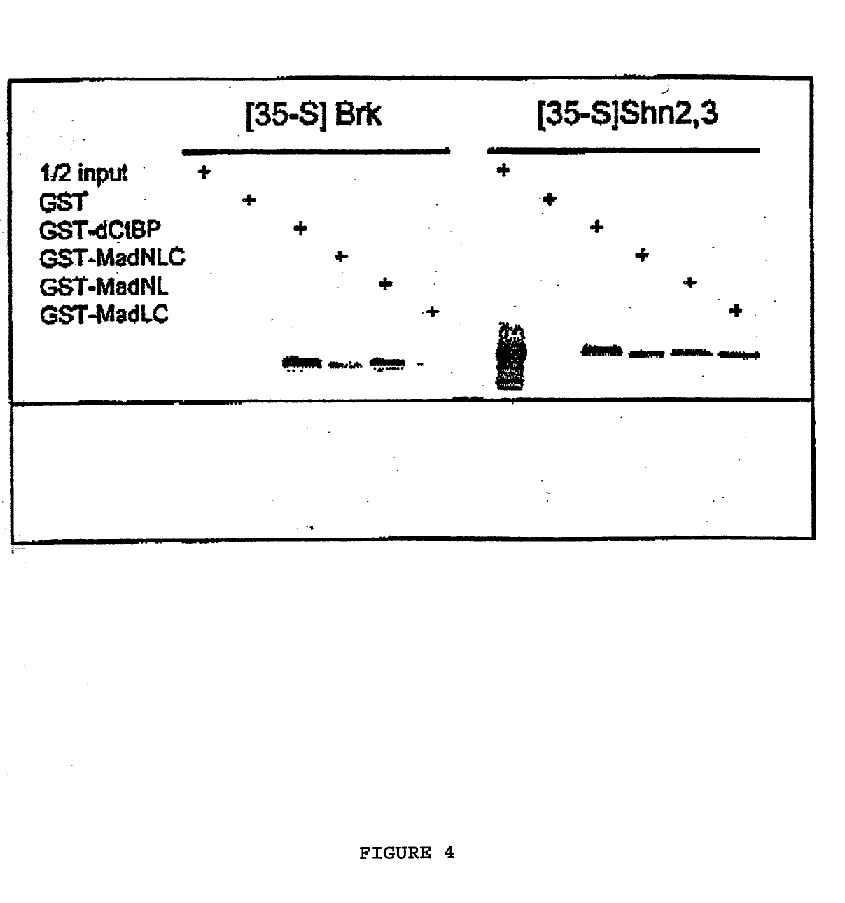

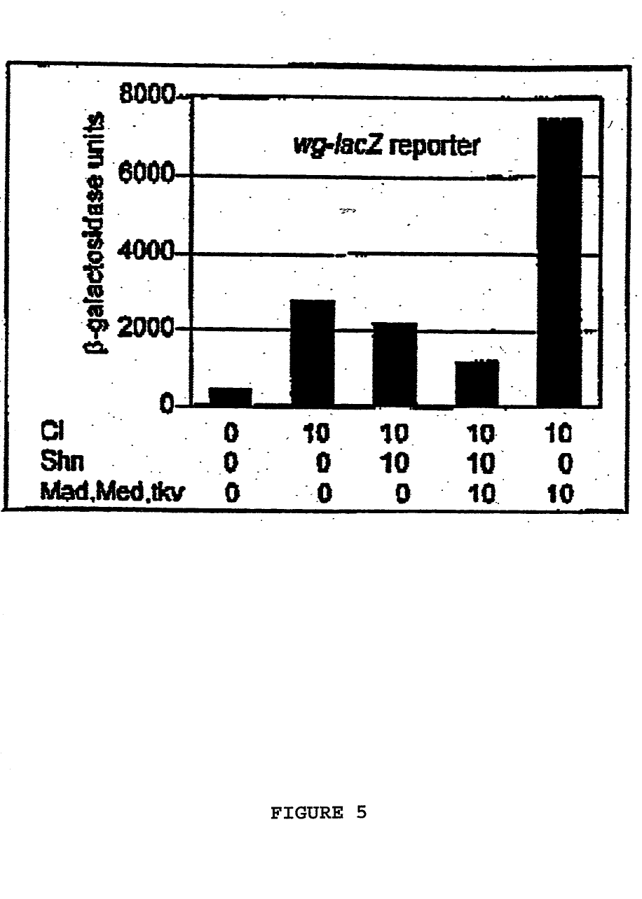

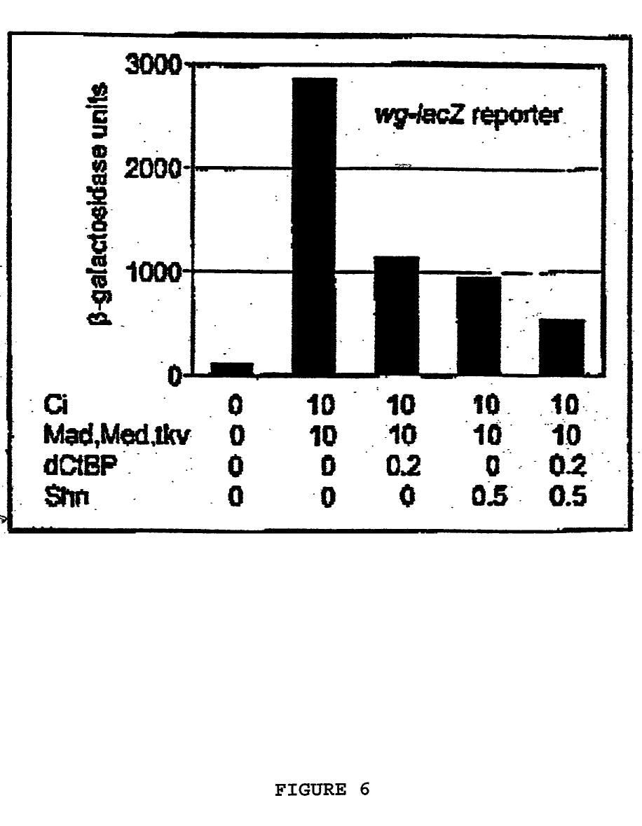

The ability of dCtBP to interact with Schnurri, a large multi-handed zinc finger protein which has been identified as a component of the Dpp pathway, was also examined. In a band-shift assay, Schnurri was shown to interact with the Mad MH2 domain (see FIG. 4). Schnurri mutants failed to undergo normal activation and repression of target genes in response to Dpp. Experiments were then performed to identify the regulatory elements that mediated Dpp repression of the Drosophila wg gene through Schnurri binding sites (see FIG. 5). Drosophila S2 cells were transfected with a reporter plasmid containing two copies of a sequence from the wingless disc enhancer region positioned next to a basal promoter driving lacZ. LacZ activity was examined and reported as production of β-galactosidase. LacZ activity was activated six-fold by co-transfection of a plasmid expressing an activated form of the transcription factor, Ci. Co-expression of Schnurri, Mad, Medea and activated tkv repressed this Ci-activated expression two-fold. Without co-expression of Schnurri, addition of Mad, Medea and tkv had the opposite effect of activating transcription. Without the presence and cooperation of Mad, Medea and tkv, Schnurri had little if any ability to repress transcription. However, the repression of lacZ in transfected cells was enhanced by co-expression of dCtBP (see FIG. 6). Using the same reporter as described above, low levels of dCtBP plasmid, 0.2 ng, were effective at repressing reporter expression with or without co-expression of Schnurri. Schnurri and CtBP are both broadly expressed and therefore are likely to be present constitutively in S2 cells. These data together support a model in which Smads, Schnurri and CtBP act in a concerted way as direct negative regulators of wingless expression.

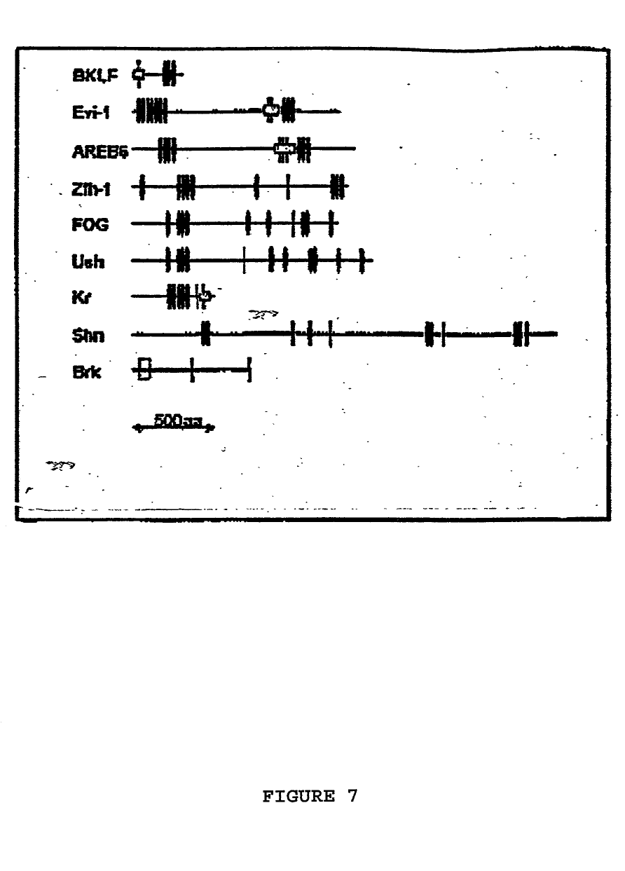

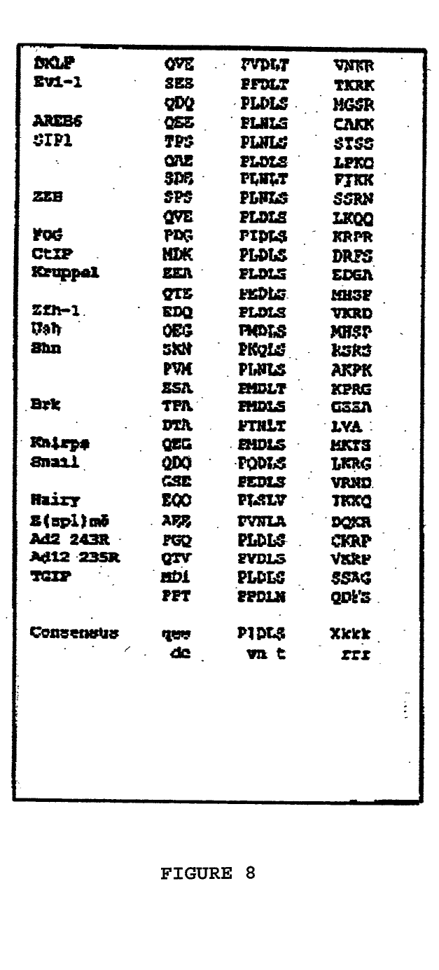

When overall structure of Schnurri is examined, it is shown to resemble several other non-homologous zinc finger proteins that are either known or suspected of interacting with CtBP (see FIGS. 7 and 8). Evi-1 inhibits TGF-β signaling by interacting directly with Smad3 (Kurokawa et al. 1998. Nature 394:92-96). Sequences closely matching the CtBP interaction motif are also found in the Smad co-repressors TGIF and SIP1.

Using a Drosophila system, it has also been found that signaling by the TGF-β family member, Dpp, is negatively regulated by the brinker (brk) gene. Dpp has the ability to trigger activation of target genes at different thresholds, allowing cells to interpret position along the Dpp gradient. The best understood example is the wing primordia of Drosophila, where spalt (sal), optomotor blind (omb), and vestigial (vg) are expressed in progressively wider patterns in response to different thresholds for activation by a Dpp gradient that emanates from the edge of the anterior compartment. Like all TGF-β pathways, the Dpp pathway activates the Smad protein Mad, which forms a complex with its partner Smad, Medea, and regulates nuclear targets such as vg, Ubx, and tinman (tin). It is known that the vertebrate BMP-responsive Smad proteins act through a Mad-like mechanism (Ishida, W. et al. 2000. J. Biol. Chem. 275:6075-6079; Kusanagi, K. et al. 2000. Mol. Biol. Cell 11:555-565). The data now show that the brinker protein (Brk) binds and represses the Dpp response elements of vg and Ubx. Thus, the previously identified Mad sites are composite Mad/brk sites. Additionally, it has been found that Brk interacts directly with Mad but that Brk is also capable of active repression when juxtaposed to an activator that is unrelated to Smads.

Experiments were performed to determine whether brk represses Dpp response elements, in particular the MD2 element. Drosophila S2 cells were co-transfected with the 2xMD2-lacZ reporter gene and varying amounts of effector plasmids that express brk, Mad, Med, and an activated version of the type I Dpp receptor, thickveins (tkvQD). Reporter expression was induced approximately four-fold by expression of Mad, Med and tkvQD. This activation of expression was blocked, however, by co-transfecting cells with 10 ng of pPac-brk plasmid. Levels of pPac-brk up to 0.4 ng had no effect. Then, using the S2 system, the effect of Brk on expression of a lacZ reporter driven by two copies of a 30 bp fragment from the Dpp-response element of Ubx was examined. Results showed that 2xUbx-lacZ was repressed by 0.08 ng of ppac-brk, indicating that in transfected S2 cells, Ubx is 100-fold more sensitive that vg to repression by Brk. These data are consistent with the expectation that sensitivity to Brk will increase as sensitivity to Dpp decreases.

In a DNA binding assay, an MBP-Brk fusion protein truncated just C-terminal to the putative heterodomain (BrkHD), which is located near its amino terminus, retained DNA binding activity and the ability to inhibit binding of Mad.

Binding of purified MBP-Brk fusion proteins to the Dpp response elements of vg and Ubx, as described above, led to experiments that compared these elements for sequence similarity that would be a Brk binding site or sites. Comparison revealed no obvious similarity outside of the previously identified Mad binding sites. However, the right-hand Mad site of Ubx overlaps an inverted repeat of the sequence GGCGCT. The vg element, for which MBP-Brk has weaker affinity, contains imperfect copies of this repeat as part of its Mad binding site. MBP-BrkHD was tested for the ability to bind one or two copies of this sequence in DNA probes that are otherwise divergent in sequence from the Ubx and vg elements. While the single copy probe was bound only weakly, MBP-BrkHD had a high affinity for the inverted repeat arrangement of the two-copy probe. The strength of this interaction in comparison to weaker binding to the Ubx probe indicated diminished affinity when a GGCGCT sequence is located near the end of the DNA probe. In addition to the strong two-copy band shift, there was a weaker monomer band for all three probes, while the Ubx probe had a third, slower mobility band that may be indicative of the presence of an additional degenerate site.

To examine the sequence specificity of Brk binding, a single copy of the site (positioned 9 bp from each end) was tested for the effects of single base pair substitutions. At least one substitution at each position caused a reduction in binding affinity of 5-fold or more, up to 20-fold, while the smallest effect was a 3-fold reduction. These data indicated that Brk makes base-specific contacts across the entire GGCGCT sequence.

To determine whether the Brk binding sites identified using the band shift assay were actually required for repression, the Ubx element was mutated to disrupt Brk binding. Each of three GGCGCT sequences (SEQ ID NO.: 2) were mutated to GTCG (SEQ ID NO.: 3) or to GGCGA (SEQ ID NO.: 4). Both mutations resulted in a significant reduction in Brk binding, but still allowed Mad to bind with nearly normal affinity. Introduction of the same triple-substitutions into the 2xUbx-lacZ reporter resulted in approximately a 100-fold decrease in sensitivity to repression by co-transfected pPac-Brk. These results demonstrated that Brk binding sites were required for repression and confirmed that the sequence specificity characterized in gel-shift experiments was also observed in cells. These results also indicated that the inhibition of MadNL-DNA interaction by Brk observed in band shift assays is not sufficient for repression in vivo.

To determine whether Brk was capable of functioning as an active repressor, Brk binding sites were positioned adjacent to sites for the unrelated Notch-responsive activator, Suppressor of Hairless (Su(H)), and reporter expression was monitored in response to co-transfected Brk. Brk completely blocked activation by Su(H) and had only a modest effect on a control reporter containing only Su(H) sites. Thus, Brk was capable of functioning as an active repressor and its ability to repress is not likely limited to targets with overlapping Mad sites.

These data considered together demonstrate that Brk acts on Dpp targets by binding to functional Mad sites. Mutational analysis revealed that Brk and Mad compete for binding to overlapping sites but that the sequence specificity of Brk is distinct from that of Smad proteins. While Brk and Mad function were shown to be intertwined by an overlapping sequence specificity, evidence has been provided that Brk is capable of functioning independently of Mad.

With identification of the mechanism of the present invention method of direct interaction of Smad proteins with co-repressors, assays can be developed, for example to identify proteins or small molecules that interact with Smad proteins to prevent interaction of CtBP with Smads or with DNA-binding co-repressors (e.g., Evi-l, TGIF, SIP1 or Schnurri), or of formation of a DNA-bound complex containing Smads, CtBP and DNA-binding co-repressors, and thus prevent repression of genes that are negatively regulated by TGFβ signaling pathways, or to identify and clone genes that are directly and negatively regulated by TGF-β signaling pathways. The compounds or genes identified through such assays would be useful in the development of drugs and therapeutics for treatment of cancer, autoimmune diseases, and other hereditary diseases that involve negative regulation by TGF-β pathways.

In one embodiment, the present invention is an in vitro assay to detect interactions between Smad proteins and the co-repressor protein CtBP, and/or DNA binding co-repressors. The assay may be an assay which detects binding of fluorescently tagged CtBP to immobilized Smad proteins or Smad cofactors. In another embodiment, the assay of the invention is a cell-based reporter assay which could be used to screen for compounds that disrupt CtBP-dependent repression by TGF-β in vivo. Such an assay would use sensitive luminescent substrates for luciferase or β-galactosidase to detect changes in TGF-β-dependent reporter expression in response to specific compounds. Both types of assays, both in vitro and in vivo, would be used to screen chemical diversity libraries. Compounds active in these screening assays would then be used in testing for pharmacological activity in animals, including humans. Such additional screening of identified compounds is a routine part of the drug development process and one of skill would understand how to use compounds identified by the methods of the present invention.

The following non-limiting examples are provided to further illustrate the present invention.

### EXAMPLES

### Example 1

### Plasmid Descriptions

All reporter plasmids were based on the vector hslacCasper. VgMD2-lacZ contained two tandem copies of the core Dpp response element listed below:

| | |
|---|---|
| TAGCCTGCCGTCGCGATTCGACAACTTTGGCCGGCACGTTGGCGAGTGTGCCATGC | (SEQ ID NO.: 5) |
| ATGCTGATGA. | |

The two copies were separated by a 25 base pair linker and inserted between the BamH1 sites of hsplaccasper.

The 2XUbx-lacZ reporter contained two tandem copies of the core Dpp response element as follows:

AATTGGACTGGCGTCAGCGCCGGCGCTG (SEQ ID NO.: 6).

The copies were inserted between the EcoR1 and Kpn1 sites of hsplaccasper.

The 2XUbxMii and 2XUbxM13 plasmids were identical to 2XUbx except for the base substitutions indicated in FIG. 5.

The 3XUbx plasmid contained 3 inverted copies of the sequence listed below:

TCTTTCTGGACTGGCGTCAGCGCCGGCGCTCT (SEQ ID NO.: 7).

The copies were inserted between the EcoR1 and Not1 sites of hsplacCasper.

LRR-lacZ and RLL-lacZ construction have been described by Johnson et al. (Johnson, K. et al. 1999. J. Biol. Chem. 274:20709-20726).

The 4XSu(H)-lacZ plasmid contained the synthetic sequence below:

| | |
|---|---|
| AATTGTTCTCACGGATCCAAAGGTTCTCACGAGATCTGTTCTCACGGATCCAAAGG | (SEQ ID NO.: 8) |
| TTCTCACGGAATTCGGATC. | |

The copies were inserted between the EcoR1 and Kpn1 sites of hsplaccasper.

The 6XBrk4XSu(H)-lacZ plasmid was derived from 4XSu(H)-lacZ by insertion between EcoR1 and Kpn1 sites of the synthetic sequence below:

AATTAGCACCGGCGCTGTACAGCGCCGGCGCTAATTAGCGCCGGCGCTGTAC (SEQ ID NO.: 9).

Effector plasmids for activated Tkv, Brk, Smad3, Smad4, activated ActR1B, Su(H) and activated Notch were based on the actin5C promoter vector pPacPL.

Effector plasmids for Mad and Med were based on the metallothionine promoter vector PMK33 as described (Maduzia, L. L. and R. W. Padgett. 1997. Biochem. Biophys. Res. Commun. 238:595-598).

The pPac-Brk plasmid was generated by cloning a 3.4 kb Brk cDNA between the BamH1 and Not1 sites of pPacPL. In addition to the Brk coding region, the resulting plasmid contains approximately 500 base pairs of the 5′-untranslated region and approximately 800 base pairs of the 3′-untranslated region

The pPac-Su(H) plasmid contained a full-length Suppressor of Hairless cDNA cloned into the polylinker of pPacPL. The pPacNact plasmid contained a Notch cDNA fragment encoding the intracellular domain, cloned into the polylinker of pPacPL.

To create a MBP-Brk fusion protein, PCR was used to position an EcoR1 site 3 base pairs upstream of the Brk initiator ATG. The resulting clone was used to create MBP-Brk by insertion between the EcoR1 and Hind3 sites of pMAL2C.

MBP-BrkHD was generated by deleting sequences between a Pst1 site 517 base pairs downstream from the Brk ATG and a Hind3 site at the 3T-end of the cDNA.

### Example 2

### Transfections and Reporter Assays

Transfections of Drosophila S2 cells and β-galactosidase reporter assays using the chromagenic substrate, CPRG, were carried out as described by (Johnson, K. et al. 1999. J. Biol. Chem. 274:20709-20716). In addition, some β-galactosidase assays were done using the chemiluminescent Gal-Screen Assay (Tropix, Inc. Bedford, Mass.) according to the supplier's instructions. For sets of transfections involving use of cDNA's cloned into the pMK33 metallothionine vector, 0.3 mM CUSO₄ was added to the cultures 24 hours after transfection. Standard deviations were calculated from the results of triplicate assays.

### Example 3

### Purification of Fusion Proteins and Band Shift Assays

MBP-Brk fusion proteins were expressed, affinity-purified using amylose-agarose, and used in band shift assays as described by Johnson et al. (Johnson, K. et al. 1999. J. Biol. Chem. 274:20709-20716), except the binding buffer was modified to be 6% ficoll, 40 mM Tris (pH 7.5), 0.2 M NaCl, 2 mM dithiothreitol, 0.02% Nonidet P-40, 1 mg/ml bovine serum albumin, and 0.04% green food coloring. A smaller format was also used, 5% polyacrylamide gels of the dimensions 10×8×0.15 cm.

### Example 4

### GST Pull-Down Assay

Radiolabelled Brk was prepared using a TNT in vitro translation kit (Promega Corp., Madison, Wis.) with ³²S-labeled methionine and a Brk cDNA cloned in Bluescript. E. coli extracts containing GST-MadNLC (full-length Mad), GST-MadNL (Mh1 and linker regions), GST-MadLC (linker and MH2 regions), and GST-dCtBP were prepared as described by Kim, J. et al. 1997. Nature 388:304-308). Adsorption of GST-fusion proteins to glutathione-sepharose followed by use of these preparations in a pull-down assay to characterize affinity for ³²S-Brk was carried out as described by Nibu et al. (Nibu, Y. et al. 1998. Science 280:101-104).

What is claimed is:

1. A method for identifying compounds that directly interact with a Smad protein or a Smad protein co-repressor to prevent protein-protein or protein-DNA interactions required for repression of transcription induced by TGF-β, activin or bone morphogenetic protein signaling in cells comprising:
(a) determining a first level of transcription detected in cells in the presence of a Smad protein and a CtBP protein before addition of a test compound;
(b) contacting said cells with the test compound; and
(c) determining a second level of transcription detected in cells in the presence of a Smad protein and a CtBP protein after addition of the test compound, wherein a decrease in the level of repression of transcription induced by the presence of the Smad protein and the CtBP protein is indicative of the ability of the test compound to interfere with transcriptional repression and to prevent repression of transcription that is produced by a TGF-β, activin, or bone morphogenetic protein signal in cells.

2. The method of claim 1 wherein transcription levels both before and after addition of the test compound are detected in cells in the presence of a Smad protein, a CtBP protein, and a co-repressor protein selected from the group consisting of Evi-1, TGIF, SIP1, and Schnurri.

3. The method of claim 1 wherein the Smad protein is Drosophila Mad or Medea.

4. The method of claim 1 wherein the CtBP protein is dCtBP, CtBP2 or any homologue of CtBP.

5. A composition identified by the method of claim 1.

6. A method for identifying a candidate gene that is directly and negatively regulated by TGF-β signaling pathways through a CtBP protein comprising:
(a) determining a first level of TGFβ-regulated target gene expression in the presence of CtBP;
(b) determining a second level of TGFβ-regulated target gene expression in the absence of the CtBP protein; and
(c) comparing the first level of expression with the second level of expression, wherein dependence of TGFβ-regulated gene expression on the presence of the CtBP protein is indicative of the ability of the candidate gene to be directly and negatively regulated by CtBP working in conjunction with the Smad protein.

7. The method of claim 6 wherein the CtBP protein is dCtBP.

8. The method of claim 6 wherein the Smad protein is Drosophila Mad or Medea.
